(19) United States
(12) Patent Application Publication
(10) Pub. No.: US 2006/0204471 A1
(43) Pub. Date: Sep. 14, 2006

(54) Compositions and regime/regimen for treating the adverse signs of cutaneous skin aging

(75) Inventors: Olivier de Lacharriere, Paris (FR); Stephanie Nouveau, Boulogne (FR)

Correspondence Address: BUCHANAN, INGERSOLL & ROONEY PC, POST OFFICE BOX 1404, ALEXANDRIA, VA, 22313-1404, US

(73) Assignee: L'OREAL, Paris (FR)

(21) Appl. No.: 11/434,207

(22) Filed: May 16, 2006

Related U.S. Application Data
(62) Division of application No. 10/156,130, filed on May 29, 2002.

(30) Foreign Application Priority Data
May 29, 2001 (FR) 01/06979

(51) Int. Cl. A61K 36/81; A61K 8/97

(52) U.S. Cl. 424/74; 424/760

(57) ABSTRACT

Cosmetic/dermatological compositions suited for combating the adverse signs of cutaneous skin aging, notably for maintaining and/or enhancing the thickness of the skin, particularly the thickness of the epidermis, comprise a thus-effective amount of at least one substance/species which is active with respect to the trophicity of the cutaneous nerves and/or at least one neurostimulant and/or at least one neuroactivator and/or at least one neuromediator, formulated into a cosmetically/dermatologically acceptable vehicle, diluent or carrier therefor.

### CROSS-REFERENCE TO PRIORITY APPLICATION

This application is a divisional of U.S. patent application Ser. No. 10/156,130, filed May 29, 2002, which claims priority under 35 U.S.C. § 119 of FR-01/06979, filed May 29, 2001, both hereby expressly incorporated by reference.

### BACKGROUND OF THE INVENTION

1. Technical Field of the Invention

The present invention relates to novel compositions comprising, formulated into appropriate vehicle, diluent or carrier, at least one substance or species which is active with respect to the trophicity/nutrition of the cutaneous nerves and/or at least one neurostimulant and/or at least one neuroactivator and/or at least one neuromediator, particularly of sensory cutaneous nerves.

This invention also relates to the administration, whether topically or orally, of at least one substance or species which is active with respect to the trophicity of the cutaneous nerves and/or at least one neurostimulant and/or at least one neuroactivator and/or at least one neuromediator, to maintain and/or to increase the thickness of the skin, particularly the thickness of the epidermis.

2. Description of the Prior Art

In mammals in general, particularly in man, the skin is composed of two compartments, namely, a deep compartment which acts as a support, the dermis, and a compartment communicating with the external environment, the epidermis.

The natural epidermis is composed mainly of three types of cells, which are keratinocytes, melanocytes and Langerhans' cells, the vast majority being keratinocytes. Each of these cell types contributes by its specific functions to the essential role played by the skin in the body.

The dermis provides the epidermis with a solid support. It is also its source of nutrients. It is mainly composed of fibroblasts and of an extra-cellular matrix itself composed mainly of collagen, of elastin and of a substance referred to as ground substance, which components are synthesized by the fibroblast. It also comprises leukocytes, mastocytes or tissue macrophages. It is also traversed by blood vessels and nerve fibers, which thus constitute the cutaneous nervous system.

The cutaneous nervous system has sensory and neurovegetative functions. The sensory function is mediated by receptors which will convert the stimuli received into influxes. These influxes will return to the sensory nerve center of the spinal cord (spinal ganglion) and then the brain, where the information is assimilated. The receptors include mecanoreceptors, thermoreceptors and nocioceptors. The neurovegetative function makes possible the innervation of the dermal vessels, sweat glands and hair-erector muscles.

Cutaneous innervation permits varying the immune functions via neurotransmitters. Thus, because of its ubiquitous distribution, the cutaneous innervation is implicated in numerous physiological functions (thermoregulation, vasomotor function, immunomodulation or neurogenic inflammation) (Pincelli C., Fantini F., Giannetti A., “Neuropeptides and Skin Inflammation,” Dermatology, 1993, 187, 153-8).

The sensory cutaneous innervation constitutes the largest fraction of the dermal and epidermal network. The majority of the sensory nerve fibers are surrounded by Schwann cells. These fibers are of A-delta type (myelinated) or of C-type (nonmyelinated).

The intraepidermal nerve fibers are distributed up to the layers closest to the surface of the epidermis. They originate from nerve bundles of the upper dermis and appear as free endings. These are very fine branchings which can rise directly into the surface layers of the epidermis, or along a more winding route between the keratinocytes, losing their myelin sheath and their Schwann cells.

Recently, an ultrastructural study coupled to immunohistochemical labelings has confirmed the existence of nerve endings in the epidermis. These structures are encountered in the intercellular spaces of all the vital layers of the epidermis, up to those closest to the surface. They establish contacts with the cellular bodies and the cytoplasmic extensions of the keratinocytes by apposition of “membrane to membrane” type, but without any structure of synaptic appearance being observed (Hilliges M., Wang L., Johansson O., “Ultrastructural evidence for nerve fibers within all vital layers of the human epidermis,” J. Invest. Dermatol., 1995, 104, 134-7).

The neuromediators exert paracrine and autocrine effects on the cutaneous cells. In the other direction, they are the carriers of nerve information from the periphery to the central nervous system. They allow the nervous system to be continuously informed of the events which are taking place at the periphery (cutaneous sensations) and thus, in reply, to control the principal physiological functions of the skin.

Keratinocytes have receptors for substance P (Staniek V., Misery L., Peguet-Navarro J., Abello J., Doutremepuich J., Claudy A., Schmitt D., “Binding and in vitro modulation of human epidermal Langerhans cell functions by substance P,” Arch. Dermatol. Res., 1997, 289, 285-92), for vasoactive intestinal peptide (VIP), for calcitonin gene related peptide (CGRP) and for neuropeptide Y (Takahashi K. et al., “Direct effects of cutaneous neuropeptides on adenylyl cyclase activity and proliferation in a keratinocyte cell line: stimulated cyclic AMP formation by CGRP and VIP/PHM, and inhibited NPY through G protein-coupled receptors,” J. Invest. Dermatol., 1993, 101, 646-51), for gastrin-releasing peptide (GRP) (Staniek V. et al., “Expression of gastrin-releasing peptide receptor in human skin,” Acta Derm. Venereol., 1996, 76, 282-6) and for acetyl-choline (muscarinic or nicotinic) (Grando S A. et al., “Keratinocyte muscarinic acetylcholine receptors: immunolocalization and partial characterization,” J. Invest. Dermatol., 1995, 104, 95-100).

Immunohistochemical methods have demonstrated that the receptor for nerve cell growth factor (NGF) is expressed on the basal keratinocytes of normal human skin. The messenger RNAs of the high- and low-affinity receptors for NGF were detected in cultured human keratinocytes (Pincelli C. et al., “Expression and function of nerve growth factor receptor on cultured keratinocytes,” J. Invest. Dermatol., 1994, 103, 13-8). NGF induces the proliferation of the keratinocytes rather than their differentiation. This proliferation is greater than that effected by epidermal growth factor (EGF). An autocrine function of NGF could be demonstrated since the keratinocytes are also capable of synthesizing and of secreting NGF (Pincelli, “Expression and function of nerve growth factor receptor on cultured keratinocytes,” J. Invest. Dermatol., 1994, 103, 13-8).

It too is known that the thickness of the epidermis decreases during chronobiological aging. The cell divisions of the basal layer decline in number. The replacement time of the horny cells becomes longer. The maturing of these cells is imperfect and the keratinization no longer results in the creation of an even and homogeneous horny layer.

It is also known that, in the case of certain disease states, such as ichthyosis, the skin is subjected to damage due to lack of cell proliferation.

It is also known that, at menopause, cutaneous aging accelerates, that the thickness of the skin decreases and that women complain that their skin is tight and that it assumes the appearance of “dry skin”, indeed even the appearance of xerosis. It is known that hormonal deficiencies associated with menopause are accompanied by a general slowdown in cell metabolism, from which it can be assumed all the same that the effects which women experience are related in particular to a decrease in the proliferation of the keratinocytes.

The consequence of the above is, thus, that the skin becomes thinner in the course of aging. As the skin is the natural barrier to external attacks and challenges, it is logical to believe that the thicker the skin, the more the sensitivity of the latter should decrease (significant barrier effect). Conversely, the thinner the skin, the more its sensitivity should be increased (increased accessibility to the nerve endings).

### SUMMARY OF THE INVENTION

It has now unexpectedly and surprisingly been determined, contrary to conventional wisdom, that the thinner the epidermis, the more nerve sensitivity decreases.

Thus, the decrease in the thickness of the epidermis observed during aging is correlated with a decrease in cutaneous innervation, the latter having as a consequence a decrease in the supply of neuromediators and/or nerve growth factors, which might result in a decrease, indeed even in a halt, in the proliferation of the cells of the epidermis, particularly of the keratinocytes.

### DETAILED DESCRIPTION OF BEST MODE AND SPECIFIC/PREFERRED EMBODIMENTS OF THE INVENTION

More particularly according to the present invention, it has now been determined that, to combat the decrease in the thickness of the skin related to aging, particularly in the thickness of the epidermis, a composition can be administered to an individual subject in need of such treatment, for example via topical application onto the skin, comprising at least one substance or species which is active with respect to the trophicity of the cutaneous nerves and/or at least one neurostimulant and/or at least one neuroactivator, particularly of sensory cutaneous nerves.

By the term “trophicity of the nerves” is intended the maintenance of the cutaneous nerves, particularly of sensory cutaneous nerves, in a nutritional good state of viability and functioning.

By the term “substance which is active with respect to the trophicity of the nerves” is intended a substance or species which permits the nerves to retain a nutritional good state of viability and functioning.

And by the term “neurostimulant and/or neuroactivator” is intended a substance or species which increases the physiological activity of the nerves.

Thus, the present invention features maintaining, by the provision of the abovementioned substances or species, good vitality, good integrity, good activity, good proliferation, good functioning and/or good excitability in the cutaneous nerves, particularly the sensory cutaneous nerves.

Accordingly, the present invention features novel compositions comprising, in an appropriate topically applicable or orally administratable medium, at least one substance or species which is active with respect to the trophicity of the cutaneous nerves and/or at least one neurostimulant and/or at least one neuroactivator and/or at least one neuromediator.

The substance or species which is active with respect to the trophicity of the cutaneous nerves is particularly active with respect to sensory cutaneous nerves.

Exemplary substances or species which are active with respect to the trophicity of the nerves, are the growth factors, PEG and glycosaminoglycans (GAG). Exemplary growth factors include, for example, NGF or glial cell line-derived neurotrophic factor (GDNF). And exemplary of the glycosamino-glycans, is, for example, hyaluronic acid.

Preferably, the compositions of the invention comprise NGF.

Exemplary neurostimulants and/or neuroactivators include, for example, capsicosides, capsidiols, capsaxanthin, capsaicinoids, such as capsaicin, or plant extracts comprising same, such as those of paprika (Capsicum annuum), of red pepper, or of pepper (Piper nigrum).

Preferably, the compositions of the invention comprise capsaicinoids or plant extracts comprising same, more preferably capsaicin.

Exemplary neuromediators include, for example, substance P, VIP, CGRP, neuropeptide Y, GRP or acetylcholine.

Preferably, the subject compositions comprise substance P.

Of course, those substances or species which are active with respect to the trophicity of the cutaneous nerves and/or the neurostimulants and/or the neuroactivators and/or the neuromediators can be formulated into the compositions of the invention either alone, or as a mixture and in any proportion.

The amount of substances or species which are active with respect to the trophicity of the nerves and/or of neurostimulants and/or of neuroactivators and/or of neuromediators included in the compositions according to the invention depends, of course, on the particular effect desired and can therefore vary to a great extent.

To provide an order of magnitude, in the compositions according to the invention, the substance or species which is active with respect to the trophicity of the nerves and/or the growth factor and/or the neurostimulant and/or the neuroactivator can be incorporated in an amount advantageously representing from 10⁻⁸% to 5% by weight of the total weight of the composition and, preferably, in an amount representing from 10^{31 5}% to 0.5% by weight of the total weight of the composition.

The compositions of the invention can be provided in any pharmaceutical dosage form whatsoever, suited equally well for topical application to the skin and/or the mucous membranes and/or the hair and/or the nails, as well as for administration via the oral route.

Preferably, the compositions of the invention are suited for administration via the topical route onto the skin.

The compositions of the invention can be a cosmetic or dermatological. Preferably, the subject compositions are cosmetic compositions. The composition is a cosmetic composition as it is intended to improve the general cutaneous appearance of the individual to whom it is administered.

Very preferably, the compositions of the invention are cosmetic compositions suited for administration via topical application.

For administration via the oral route, the compositions of the invention can be provided in any suitable form, particularly in the form of a solution to be ingested orally, of a syrup, of a tablet, including a sugar-coated tablet, of a capsule, including a gelatin capsule, or of a nutritional food or of a nutritional supplement.

The subject compositions can additionally comprise at least one appropriate excipient suitable for oral administration.

For administration by topical application to the skin, the hair and/or the mucous membranes, the compositions according to the invention necessarily comprise a cosmetically acceptable medium (vehicle, diluent or carrier), namely, a medium compatible with the skin, the mucous membranes, the nails or the hair, and can be provided in any pharmaceutical dosage form normally used for topical application, in particular in the form of an aqueous, aqueous/alcoholic or oily solution, of an oil-in-water or water-in-oil or multiple emulsion, of an aqueous or oily gel, of a liquid, pasty or solid anhydrous product, or of a dispersion of oil in an aqueous phase using spherules, it being possible for these spherules to be polymeric nanoparticles, such as nanospheres and nanocapsules, or even better lipid vesicles of ionic and/or nonionic type.

Such compositions can be more or less fluid and can have the appearance of a white or colored cream, of an ointment, of a milk, of a lotion, of a serum, of a paste or of a foam. It can optionally be topically applied onto the skin in the form of an aerosol. It can also be provided in the solid form and, for example, in the form of a stick. It can be used as a care product, as a cleansing product, as a makeup product, or as a simple deodorant product.

In known manner, the compositions of the invention can comprise the additives and adjuvants which are standard in the cosmetic and dermatological fields, such as hydrophilic or lipophilic gelling agents, hydrophilic or lipophilic active agents or principles, preservatives, antioxidants, solvents, fragrances, fillers, UV-screening agents, pigments, chelating agents, odor absorbers and colorants. The amounts of these various adjuvants are those conventionally formulated in the fields under consideration and, for example, range from 0.01% to 20% by weight of the total weight of the composition. These additives and adjuvants, depending upon their nature, can be incorporated into the fatty phase, into the aqueous phase, into the lipid vesicles and/or into the nanoparticles.

When a composition of the invention is an emulsion, the proportion of the fatty phase can advantageously range from 5% to 80% by weight and preferably from 5% to 50% of the total weight of the composition. The oils, the emulsifiers and the coemulsifier included in the composition in the form of an emulsion are selected from among those conventionally used in the field under consideration. The emulsifier and the coemulsifier are present in the composition in a proportion advantageously ranging from 0.3% to 30% by weight and preferably from 0.5% to 20% of the total weight of the composition.

Exemplary oils which can be formulated according to the invention include the mineral oils, oils of vegetable origin (apricot oil, sunflower oil), oils of animal origin, synthetic oils, silicone oils and fluorinated oils (perfluoropolyethers). Also exemplary are the fatty substances, fatty alcohols (cetyl alcohol), fatty acids or waxes (beeswax).

Exemplary emulsifiers and coemulsifiers according to the invention include the esters of a fatty acid and of polyethylene glycol, such as PEG-40 stearate or PEG-100 stearate, or esters of a fatty acid and of a polyol, such as glyceryl stearate and sorbitan tristearate.

And exemplary hydrophilic gelling agents include carboxyvinyl polymers (carbomer), acrylic copolymers, such as acrylate/alkyl-acrylate copolymers, polyacrylamides, polysaccharides, natural gums and clays, and representative lipophilic gelling agents include the modified clays, such as bentones, metal salts of fatty acids, hydrophobic silica and polyethylenes.

The subject compositions can comprise other hydrophilic biologically active principles or agents, such as proteins or protein hydrolysates, amino acids, polyols, urea, allantoin, sugars and sugar derivatives, plant extracts and hydroxy acids.

Exemplary such lipophilic active agents or principles include retinol (vitamin A) and derivatives thereof, α-tocopherol (vitamin E) and derivatives thereof (esters, salts, and the like), essential fatty acids, ceramides, essential oils, salicylic acid and derivatives thereof, or vitamins B1, B6 and/or B12.

It is also intended to include vitamin C (or ascorbic acid) and derivatives thereof (esters, salts, and the like) in the compositions of the invention.

It is also envisaged to add thereto a plant extract rich in isoflavonoids, such as, for example, the soya bean (Glycina max) extract available from Archer Daniels Midland Company under the trademark Novasoy®.

Too, it is also envisaged to add a progestational agent, such as, for example, 17-hydroxy-progesterone or pregnenolone, to the compositions of the invention.

This invention, thus, also features formulating compositions comprising at least one substance or species which is active with respect to the trophicity of the cutaneous nerves and/or of a neurostimulant and/or of a neuroactivator and/or of a neuromediator as described above, said substance/agent or composition comprising same being well suited to maintain good vitality, good integrity, good activity, good proliferation, good functioning and/or good excitability in the cutaneous nerves.

This invention also features formulating compositions comprising at least one substance/agent which is active with respect to the trophicity of the cutaneous nerves and/or of a neurostimulant and/or of a neuroactivator and/or of a neuromediator, said substance/agent or composition comprised thereof being well suited to maintain and/or to enhance the thickness of the skin, particularly the thickness of the epidermis.

### BRIEF DESCRIPTION OF THE DRAWINGS

FIG. 1 illustrates the mean values of the thickness of the epidermis for various thresholds for detection of cold.

FIG. 2 illustrates the thermal detection (heat and cold) threshold as a function of the age group.

FIG. 3 illustrates the mean values of the total scores obtained in the capsaicin test for the various age categories.

In order to further illustrate the present invention and the advantages thereof, the following specific examples are given, it being understood that same are intended only as illustrative and in nowise limitative.

### EXAMPLE 1

Correlation Between the Decrease in Cutaneous Thermal Sensitivity and the Thickness of the Epidermis, Measured in Elderly Subjects:

Methodology:
(a) Number of subjects: 122 women aged from 60 to 80 years;
(b) Measurement of the thresholds for cutaneous detection of cold:

This measurement was carried out with the Thermal Sensory Analyzer (TSA 2001 Medoc Ltd, Ramat Yishai, Israel), the probe of which is capable, by the Peltier effect, of cooling the skin along a calibrated temperature gradient.

The threshold for sensitivity to cold was thus detected beginning from a “neutral” temperature for the skin of 32° C. It was sufficient for the subject, as soon as she experienced a change in temperature, to indicate via a pushbutton, which action automatically resulted in the thermal stimulus being halted.

A series of 5 stimuli (below 32° C.) was recorded and a mean was calculated in order to determine the detection threshold. This measurement was carried out on the face.
(c) Measurement of the thickness of the epidermis:

The thickness of the epidermis was measured by high-resolution (25 MHz) ultrasonic echography on the back of the hand (Querleux B, Lévêque J L, de Rigal J: In vivo cross sectional ultrasonic imaging of human skin.

Dermatologica, 1988, 77, 332-337).

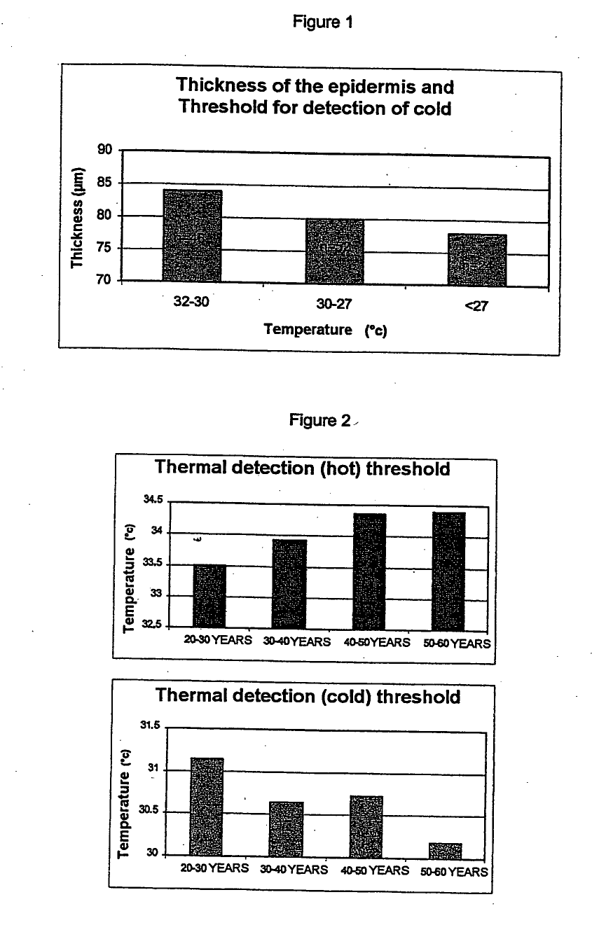

Results:
The mean values of the thickness of the epidermis for various thresholds for detection of cold are presented in FIG. 1.

The greater the detection temperature, the greater the sensitivity of the skin to cold and the greater the thickness of the epidermis. On the other hand, the lower the sensitivity of the skin to cold, the more the epidermis had atrophied.

### EXAMPLE 2

Measurements of Thermal Acuity as a Function of Age:

Methodology:
(a) Number of subjects: 160 women categorized into 4 groups of 40:
   1. 40 subjects from 20 to 30 years old,
   2. 40 subjects from 30 to 40 years old,
   3. 40 subjects from 40 to 50 years old,
   4. 40 subjects from 50 to 60 years old.
(b) Measurement of the thresholds for cutaneous detection of heat and of cold (thermal sensitivity):

This measurement was carried out according to the protocol described in Example 1 on the upper lip (cutaneous part) in the right or left side region according to the randomization plan.

Results: (FIG. 2)
The older the subjects, the less sensitive they are to temperature (decrease in the thresholds for detection of cold).

### EXAMPLE 3:

Measurements of the Sensitivity to Capsaicin as a Function of Age:

Methodology:
Subjects: 152 women aged from 18 to 65 years old: 35 were less than 30 years old; 32 were between 30 and 40 years old; 43 were between 41 and 50 years old; 20 were between 51 and 60 years old; 21 were more than 60 years old.

Capsaicin Test:
The capsaicin test employs a natural compound from the family of the alkaloids present in the fruits of various species of the belladonna family. Capsaicin is the hot substance present in the capsicums. The application of capsaicin to the skin promotes a brief release of substance P in the nerve endings, the consequence of which is the appearance of feelings of burning and of tingling, sometimes accompanied by a localized erythema at the application region. These reactions are transitory and completely reversible in the hours which follow. These reactions are directly related to sensory innervation; the greater the latter, the greater the reaction, and vice versa.
(i) Application of 20 mg/cm² of Zostrix HP (cream comprising 0.075% of capsaicin) to a site of 4 cm² at the angle of the jaw.
(ii) Gradings (scale with 4 levels) by the subject of the sensations of tingling, inflammation and itching experienced at 3 min, 5 min, 10 min, 15 min, 20 min, 25 min and 30 min after the application of capsaicin according to the following scale:
0=Absence of reaction;
1=Slight or doubtful feeling;
2=Moderate or clearcut feeling;
3=Strong or significant feeling.
   (iii) Clinical evaluations of the erythema by the investigator at 3 min, 5 min, 15 min, 20 min, 25 min and 30 min according to the following scale:
0=Absence of reaction;
1=Slight or doubtful erythema;
2=Moderate erythema localized at the application region;
3=Significant erythema with extension beyond the limits of the application region.

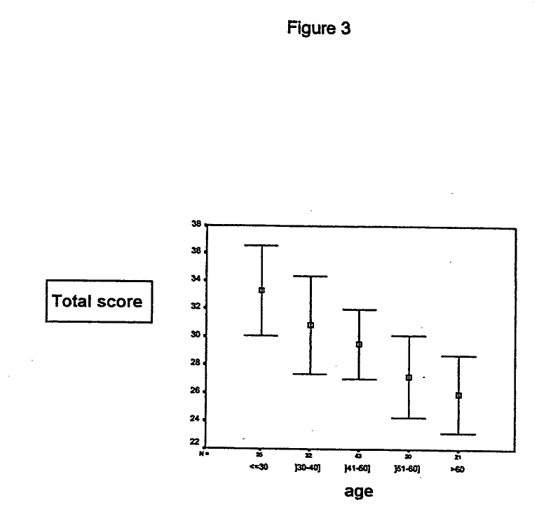

Results (See FIG. 3):
These results indicate a marked reduction in the scores for the various age categories.

The three preceding examples clearly evidence a reduction in the cutaneous sensitivity correlated with a decrease in the thickness of the epidermis during aging. The first example furthermore evidences a decrease in the thickness of the epidermis correlated with a loss in sensitivity to cold.

### EXAMPLE 4

This example is of specific compositions/formulations according to the invention, prepared via simple admixing of the various components.

Composition 1—Skin Cream:

| | |
|---|---|
| Capsaicin | 0.0005 |
| Glyceryl stearate | 2.00 |
| Polysorbate 60 (Tween 60, marketed by ICI) | 1.00 |
| Stearic acid | 1.40 |
| Glycyrrhetinic acid | 2.00 |
| Triethanolamine | 0.70 |
| Carbomer | 0.40 |
| Liquid fraction of karite butter | 12.00 |
| Sunflower oil | 10.00 |
| Antioxidant | 0.05 |
| Fragrance | 0.5 |
| Preservative | 0.30 |
| Water | q.s. for 100% |

Composition 2—Skin Cream:

| | |
|---|---|
| Capsaicin | 0.0001 |
| 17-OH-Progesterone or pregnenolone acetate | 1 |
| Glyceryl stearate | 2.00 |
| Polysorbate 60 (Tween 60, marketed by ICI) | 1.00 |
| Stearic acid | 1.40 |
| Glycyrrhetinic acid | 2.00 |
| Triethanolamine | 0.70 |
| Carbomer | 0.40 |
| Liquid fraction of karite butter | 12.00 |
| Sunflower oil | 10.00 |
| Antioxidant | 0.05 |
| Fragrance | 0.5 |
| Preservative | 0.30 |
| Water | q.s. for 100% |

Composition 3—Skin Cream:

| | |
|---|---|
| PEG | 8.00 |
| Glyceryl stearate | 2.00 |
| Polysorbate 60 (Tween 60, marketed by ICI) | 1.00 |
| Stearic acid | 1.40 |
| Glycyrrhetinic acid | 2.00 |
| Triethanolamine | 0.70 |
| Carbomer | 0.40 |
| Liquid fraction of karite butter | 12.00 |
| Sunflower oil | 10.00 |
| Antioxidant | 0.05 |
| Fragrance | 0.5 |
| Preservative | 0.30 |
| Water | q.s. for 100% |

Composition 4—Skin Cream

| | |
|---|---|
| Capsicum annuum extract | 0.005 |
| Glyceryl stearate | 2.00 |
| Polysorbate 60 (Tween 60, marketed by ICI) | 1.00 |
| Stearic acid | 1.40 |
| Glycyrrhetinic acid | 2.00 |
| Triethanolamine | 0.70 |
| Carbomer | 0.40 |
| Liquid fraction of karite butter | 12.00 |
| Sunflower oil | 10.00 |
| Antioxidant | 0.05 |
| Fragrance | 0.5 |
| Preservative | 0.30 |
| Water | q.s. for 100% |

Composition 5—Skin Cream

| | |
|---|---|
| Hyaluronic acid | 2.00 |
| PEG | 5.00 |
| Glyceryl stearate | 2.00 |
| Polysorbate 60 (Tween 60, marketed by ICI) | 1.00 |
| Stearic acid | 1.40 |
| Glycyrrhetinic acid | 2.00 |
| Triethanolamine | 0.70 |
| Carbomer | 0.40 |
| Liquid fraction of karite butter | 12.00 |
| Sunflower oil | 10.00 |
| Antioxidant | 0.05 |
| Fragrance | 0.5 |
| Preservative | 0.30 |
| Water | q.s. for 100% |

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A cosmetic/dermatological composition suited for combating the signs of cutaneous skin aging, comprising a thus-effective amount of at least one neurostimulant and/or neuroactivator, formulated into a cosmetically/dermatologically acceptable vehicle, diluent or carrier therefor.

2. The cosmetic/dermatological composition as defined by claim 1, wherein said at least one neurostimulant and/or neuroactivator is selected from the group consisting of capsicosides, capsidiols, capsaxanthin, capsaicinoids, and plant extracts comprising same.

3. The cosmetic/dermatological composition as defined by claim 2, wherein said at least one neurostimulant and/or neuroactivator comprises a capsaicinoid or plant extract comprised thereof.

4. The cosmetic/dermatological composition as defined by claim 3, comprising capsaicin.

5. The cosmetic/dermatological composition as defined by claim 3, comprising an extract of paprika (Capsicum annuum), of red pepper, or of pepper (Piper nigrum).

6. The cosmetic/dermatological composition as defined by claim 1, comprising from 10⁻⁸% to 5% by weight of said at least one neurostimulant and/or neuroactivator.

7. The cosmetic/dermatological composition as defined by claim 6, comprising from 10⁻⁵% to 0.5% by weight of said at least one neurostimulant and/or neuroactivator.

8. The cosmetic/dermatological composition as defined by claim 1, formulated for topical application onto the skin, mucous membranes, hair and/or nails.

9. The cosmetic/dermatological composition as defined by claim 1, formulated for oral administration.

10. A regime or regimen for maintaining beneficial vitality; integrity, activity, proliferation, functioning and/or excitability in the cutaneous nerves of an individual subject in need of such treatment, comprising administering thereto a thus-effective amount of at least one neurostimulant and/or neuroactivator, formulated into a cosmetically/dermatologically acceptable vehicle, diluent or carrier therefor.

11. A regime or regimen for maintaining and/or enhancing the thickness of the skin of an individual subject in need of such treatment, comprising administering thereto a thus-effective amount of at least one neurostimulant and/or neuroactivator, formulated into a cosmetically/dermatologically acceptable vehicle, diluent or carrier therefor.

12. A regime or regimen for maintaining and/or enhancing the thickness of the epidermis of an individual subject in need of such treatment, comprising administering thereto a thus-effective amount of at least one neurostimulant and/or neuroactivator, formulated into a cosmetically/dermatologically acceptable vehicle, diluent or carrier therefor.

13. The regime or regimen as defined by claim 10, wherein said at least one neurostimulant and/or neuroactivator is selected from the group consisting of capsicosides, capsidiols, capsaxanthin, capsaicinoids, and plant extracts comprising same.

14. The regime or regimen as defined by claim 11, wherein said at least one neurostimulant and/or neuroactivator is selected from the group consisting of capsicosides, capsidiols, capsaxanthin, capsaicinoids, and plant extracts comprising same.

15. The regime or regimen as defined by claim 12, wherein said at least one neurostimulant and/or neuroactivator is selected from the group consisting of capsicosides, capsidiols, capsaxanthin, capsaicinoids, and plant extracts comprising same.

16. The regime or regimen as defined by claim 10, comprising topically applying said at least one neurostimulant and/or neuroactivator onto the skin, mucous membranes, hair and/or nails of said individual subject.

17. The regime or regimen as defined by claim 11, comprising topically applying said at least one neurostimulant and/or neuroactivator onto the skin, mucous membranes, hair and/or nails of said individual subject.

18. The regime or regimen as defined by claim 12, comprising topically applying said at least one neurostimulant and/or neuroactivator onto the skin, mucous membranes, hair and/or nails of said individual subject.

19. A regime or regimen for combating the decrease in the thickness of the skin related to aging, comprising administering to an individual in need of such treatment, the thickness of whose skin has diminished with age, a thus-effective skin-thickening amount of at least one neurostimulant and/or neuroactivator.

20. A regime or regimen for combating the decrease in the thickness of the skin relating to aging and/or the diminishing sensitivity of the nerves therein, comprising administering to an individual in need of such treatment, the thickness of whose skin and/or sensitivity of whose nerves therein have diminished with age, a thus-effective skin-thickening and/or nerve sensitivity-increasing amount of at least one neurostimulant and/or neuroactivator.
